(19) United States
(12) Patent Application Publication
(10) Pub. No.: US 2026/0256859 A1
(43) Pub. Date: Sep. 3, 2026

(54) COMPOSITION FOR PREVENTING, AMELIORATING, OR TREATING TUBERCULOSIS COMPRISING EXTRACT OF PHILADELPHUS SP. PLANT AS ACTIVE INGREDIENT

(71) Applicants: KOREA INSTITUTE OF ORIENTAL MEDICINE, Daejeon (KR); CHUNGNAM NATIONAL UNIVERSITY HOSPITAL, Daejeon (KR); THE INDUSTRY & ACADEMIC COOPERATION IN CHUNGNAM NATIONAL UNIVERSITY (IAC), Daejeon (KR)

(72) Inventors: CHANG-HWA SONG, Daejeon (KR); JI-AE CHOI, Daejeon (KR); TAM DOAN NGUYEN, Daejeon (KR); DONG-SEON KIM, Daejeon (KR); HEUNG JOO YUK, Daejeon (KR); EUNJUNG SON, Sejong-si (KR); YUN MI LEE, Daejeon (KR); YOON-YOUNG SUNG, Daejeon (KR)

(21) Appl. No.: 19/490,002

(22) PCT Filed: Jun. 21, 2024
(86) PCT No.: PCT/KR2024/008605

§ 371 Date: Dec. 4, 2025

(30) Foreign Application Priority Data
Jun. 21, 2023 (KR) 10-2023-0079428

(51) Int. Cl. A61K 36/185; A61K 31/133; A61K 31/4409; A61K 31/496; A61P 31/06

(52) U.S. Cl. CPC A61K 36/185; A61K 31/133; A61K 31/4409; A61K 31/496; A61P 31/06; A61K 2236/333; A61K 2236/51

(57) ABSTRACT

A composition for preventing, ameliorating, or treating tuberculosis includes an extract of Philadelphus sp. plant as active ingredient. The extract of Philadelphus sp. plant significantly reduces the number of Mycobacterium tuberculosis not only in mouse bone marrow-derived macrophages but also in mouse (i.e., in vivo sample) infected with Mycobacterium tuberculosis, and it exhibits a synergistic effect on reducing the number of Mycobacterium tuberculosis when co-administered with an anti-tuberculosis agent. Therefore, the extract of Philadelphus sp. plant of the present invention can be advantageously used as an anti-tuberculosis agent.

### BACKGROUND

### 1. Technical Field

The present invention relates to a composition for preventing, ameliorating, or treating tuberculosis including an extract of Philadelphus sp. plant as active ingredient.

This study was supported by the project for the development of new natural product for tuberculosis treatment at Chungnam National University Hospital (2022.06.01-2023.05.31), and by the major project of the Korea Institute of Oriental Medicine under (or affiliated with) the Ministry of Science and ICT, South Korea titled “Establishment of Safety and Efficacy of Herbal Prescriptions at Korean Medicine Institutions” (Project numbers: 1711195899, KSN1823310).

### 2. Background Art

Tuberculosis (TB) is an infectious disease caused by infection with Mycobacterium, particularly Mycobacterium tuberculosis. It is estimated that one-third of the world's population is infected with Mycobacterium tuberculosis, and approximately 8 million new cases occur annually. Most infected individuals are asymptomatic, but about one-tenth of them develop the disease. Without proper treatment, more than half of those who develop active TB may die.

In developed countries, the tuberculosis infection rate is below 0.1%, and the disease incidence rate is under 25%. In South Korea, the prevalence of active pulmonary tuberculosis (as diagnosed by chest X-ray) decreased from 5.1% in 1965 to 1.0% in 1995. The prevalence of smear-positive tuberculosis also declined from 0.94% to 0.22%, and the annual risk of tuberculosis infection dropped from 5.3% to 0.5%. The mortality rate per 100,000 population fell from 10.4 in 1991 to 6.3 in 2001. However, tuberculosis still remains one of the top ten causes of death.

The six-month short-course treatment regimen involves the combined administration of the first-line anti-tuberculosis agents rifampicin, isoniazid, pyrazinamide, and ethambutol for two months, followed by a four-month treatment with three drugs—rifampin, isoniazid, and ethambutol. During the initial two-month intensive treatment phase, streptomycin may be used in place of ethambutol. In such case, the maintenance therapy involves the administration of rifampin and isoniazid.

However, all of these antibiotics were developed more than 50 years ago, and the development of new therapeutic agents has not been active. Moreover, strains of Mycobacterium tuberculosis resistant to multiple drugs (MDR; multi-drug resistant) and extensively resistant to drugs (XDR; extensively-drug resistant) have emerged, leading to a gradual decline in the effectiveness of treatments using conventional therapeutic agents. Therefore, research is needed into new anti-tuberculosis agents or substances that can enhance the susceptibility of Mycobacterium tuberculosis to existing treatments.

As for prior technologies related to tuberculosis treatments, Korean Patent Registration No. 1833048 discloses a pharmaceutical composition for treating tuberculosis including colinin and a method for its preparation. Further, Korean Patent Registration No. 0656969 discloses a pharmaceutical composition and a functional health food for treating tuberculosis including an extract of Zanthoxylum fruit as active ingredient. However, there has been no disclosure of a composition for preventing, ameliorating, or treating tuberculosis including an extract of Philadelphus sp. plant—specifically, an extract of Philadelphus schrenkii (i.e., thin-leaved Philadelphus sp.)—as active ingredient, as proposed in the present invention.

### SUMMARY

The present invention is devised in view of the above-mentioned needs, and provide by the present invention is a composition for preventing, ameliorating, or treating tuberculosis including an extract of Philadelphus sp. plant as active ingredient. The extract of Philadelphus sp. plant significantly reduces the number of Mycobacterium tuberculosis not only in mouse bone marrow-derived macrophages but also in mouse (i.e., in vivo sample) infected with Mycobacterium tuberculosis, and it exhibits a synergistic effect on reducing the number of Mycobacterium tuberculosis when co-administered with an anti-tuberculosis agent. Based on this finding, the present invention is completed.

To solve the above problem, the present invention provides a pharmaceutical composition for prevention or treatment of tuberculosis including an extract of Philadelphus sp. plant as active ingredient.

The present invention further provides a functional health food composition for prevention or amelioration of tuberculosis including an extract of Philadelphus sp. plant as active ingredient.

The present invention further provides a feed additive for prevention or amelioration of tuberculosis including an extract of Philadelphus sp. plant as active ingredient.

The present invention further provides a veterinary composition for prevention or treatment of tuberculosis including an extract of Philadelphus sp. plant as active ingredient.

The present invention further provides an oriental herbal medicine composition for anti-tuberculosis including an extract of Philadelphus sp. plant as active ingredient.

The present invention still further provides a method for suppressing growth of Mycobacterium tuberculosis by administering an extract of Philadelphus sp. plant alone or in combination with an anti-tuberculosis agent to an animal other than human.

The present invention relates to a composition for preventing, ameliorating, or treating tuberculosis including an extract of Philadelphus sp. plant as active ingredient. The extract of Philadelphus sp. plant significantly reduces the number of Mycobacterium tuberculosis not only in mouse bone marrow-derived macrophages but also in mouse (i.e., in vivo sample) infected with Mycobacterium tuberculosis, and it exhibits a synergistic effect on reducing the number of Mycobacterium tuberculosis when co-administered with an anti-tuberculosis agent.

### BRIEF DESCRIPTION OF THE DRAWINGS

FIG. 1 shows the changes in the number of Mycobacterium tuberculosis (M. tuberculosis H37Rv; Rv) in mouse bone marrow-derived macrophages (BMDM) after treatment with an extract of various Philadelphus sp. plants [\textcircled{1} Philadelphus tenuifolius leaf extract, \textcircled{2} Philadelphus pekinensis Rupr. leaf extract, \textcircled{3} Philadelphus schrenkii leaf extract, \textcircled{4} Philadelphus seoulensis leaf extract, or \textcircled{5} Philadelphus schrenckii var. jackii Koehne leaf extract]. In the drawings, ‘Thin-leaved’ refers to the Philadelphus tenuifolius leaf extract treatment group, ‘Aegi’ refers to the Philadelphus pekinensis Rupr. leaf extract treatment group, ‘Gokwang’ refers to the Philadelphus schrenkii leaf extract treatment group, ‘Seoul’ refers to the Philadelphus seoulensis leaf extract treatment group, and ‘Haired’ refers to the Philadelphus schrenckii var. jackii Koehne leaf extract treatment group. ** and *** indicate that the number of M. tuberculosis in Philadelphus sp. plant leaf extract treatment group was statistically significantly reduced compared to the tuberculosis infection group (Rv, control), in which ** denotes p<0.01 and *** denotes p<0.001.

FIG. 2 shows the results of infecting mouse bone marrow-derived macrophages (BMDM) with Mycobacterium tuberculosis (M. tuberculosis H37Rv; Rv) and treating them with an anti-tuberculosis agent [\textcircled{a} Isoniazid (INH), \textcircled{b} Rifampicin (RIF), or \textcircled{c} Ethambutol (EMB)], demonstrating a reduction in the number of intracellular M. tuberculosis. ** and *** indicate that the number of M. tuberculosis in the anti-tuberculosis agent treatment groups was statistically significantly reduced compared to the tuberculosis infection group (Rv, control), in which ** denotes p<0.01 and *** denotes p<0.001.

FIG. 3 shows the results after administering Philadelphus tenuifolius leaf extract to M. tuberculosis-infected mice, showing that the number of M. tuberculosis in the lungs of the mice decreased. * indicates that the number of M. tuberculosis in the lungs of the mice treated with Philadelphus tenuifolius leaf extract of the present invention was statistically significantly reduced compared to the tuberculosis infection group, with p<0.05.

### DETAILED DESCRIPTION

The present invention relates to a pharmaceutical composition for prevention or treatment of tuberculosis including an extract of Philadelphus sp. plant as active ingredient.

In one embodiment of the present invention, any plant from the Philadelphus species (Philadelphus sp.) can be used, and preferably, one or more selected from Philadelphus tenuifolius, Philadelphus pekinensis Rupr., Philadelphus schrenkii, Philadelphus seoulensis, and Philadelphus schrenckii var. jackii Koehne are used. More preferably, Philadelphus tenuifolius (or thin-leaved Philadelphus sp.) is used, but it is not limited thereto.

The extract of Philadelphus sp. plant can be obtained from the whole parts, above-ground parts, under-ground parts, leaves, stems, or flowers of the Philadelphus schrenkii species. Preferably, the leaves are used, but it is not limited thereto.

In one embodiment of the present invention, the composition of the present invention may further include an anti-tuberculosis agent in addition to the active ingredient. Preferably, one or more anti-tuberculosis agents selected from rifampicin, isoniazid, ethambutol, and bedaquiline may be included. More preferably, the composition further includes rifampicin, isoniazid (isonicotinyl hydrazine), or ethambutol, but it is not limited thereto.

The active ingredient of the present invention has an antimicrobial activity against Mycobacterium tuberculosis H37Rv when used alone, and when co-administered with an anti-tuberculosis agent, it exhibits a synergistic effect on antimicrobial activity.

In the present invention, the synergistic effect refers to the effect obtained when a single function interacts in multiple ways. When the action of one substance is enhanced by the presence of another substance, the two substances are said to have a synergistic effect (i.e., enhancing effect). It means that the mixture cooperates to exert a better or more extended total effect than the sum of the individual effects of the components when taken independently.

Extraction solvent for the extract of Philadelphus sp. plant of the present invention may be water, C1 to C4 lower alcohol, or a mixture thereof, with ethanol being preferable, but it is not limited thereto.

The pharmaceutical composition of the present invention may further include a pharmaceutically acceptable carrier, excipient, or diluent in addition to the active ingredient.

Furthermore, the composition can be formulated into various forms such as powders, granules, tablets, capsules, suspensions, emulsions, syrups, aerosols, or oral dosage forms, topical preparations, suppositories, and sterile injectable solutions, according to conventional methods, but it is not limited thereto.

The carriers, excipients, and diluents that may be included in the composition include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oil.

When formulating, common diluents or excipients such as fillers, bulking agents, binders, humectants, disintegrants, and surfactants are used.

Solid dosage forms for oral administration include tablets, pills, powders, granules, capsules, and the like. These solid dosage forms are prepared by mixing the active ingredient with at least one excipient such as starch, calcium carbonate, sucrose, lactose, or gelatin. Additionally, lubricants such as magnesium stearate and talc are also used. Liquid dosage forms for oral administration include suspensions, solutions, emulsions, and syrups. In addition to commonly used diluents such as water and liquid paraffin, various excipients such as humectants, sweeteners, flavoring agents, and preservatives may be included.

Formulations for parenteral administration include sterile aqueous solutions, non-aqueous solvents, suspensions, emulsions, freeze-dried preparations, and suppositories. Non-aqueous solvents and suspensions may include propylene glycol, polyethylene glycol, vegetable oils such as olive oil, and injectable esters such as ethyl oleate. The base for suppositories may include Witepsol, macrogol, Tween 61, cocoa butter, lauric acid, glycerogelatin, and the like.

The appropriate dosage of the pharmaceutical composition according to the present invention may vary depending on factors such as the formulation method, administration route, the patient's age, weight, gender, pathological condition, diet, administration time, administration route, excretion rate, and response sensitivity. The concentration of the active ingredient included in the composition of the present invention can be determined based on therapeutic purposes, the patient's condition, required duration, etc., and it is not limited to a specific concentration range.

The present invention further relates to a functional health food composition for prevention or amelioration of tuberculosis including an extract of Philadelphus sp. plant as active ingredient.

The composition of the present invention may further include an anti-tuberculosis agent in addition to the active ingredient. Preferably, one or more anti-tuberculosis agents selected from rifampicin, isoniazid, and ethambutol may be included, but it is not limited thereto.

The composition is preferably manufactured in any of the forms selected from powder, granules, pills, tablets, capsules, candies, syrups, and beverages, but it is not limited thereto.

When the functional health food composition of the present invention is used as a food additive, the active ingredient can be added as is or used with other foods or food ingredients, and can be used appropriately according to conventional methods. The amount of the active ingredient to be mixed can be suitably determined depending on its intended use (prevention, health, or therapeutic treatment). Generally, when manufacturing food or beverages, the composition of the present invention is added in an amount of 15 parts by weight or less, preferably 10 parts by weight or less, relative to the raw material. However, when consumed for long-term health and hygiene purposes or health management purposes, the amount may be less than the above range, and since there are no safety issues, the active ingredient can also be used in amounts greater than the above range.

There is no particular limitation on the types of food to which the active ingredient can be added. Examples of foods that can contain the active ingredient include meat, sausages, bread, chocolate, candies, snacks, cookies, pizza, ramen, other noodles, chewing gum, dairy products including ice cream, various soups, beverages, tea, drinks, alcoholic beverages, vitamin complexes, and all types of functional health foods in the conventional sense.

When the composition of the present invention is used as a health beverage, it may contain various flavoring agents or natural carbohydrates as additional ingredients, just like conventional beverages. The natural carbohydrates mentioned above include monosaccharides such as glucose and fructose, disaccharides such as maltose and sucrose, polysaccharides such as dextrins and cyclodextrins, and sugar alcohols such as xylitol, sorbitol, and erythritol. As sweeteners, natural sweeteners like thaumatin and stevia extract, or synthetic sweeteners such as saccharin and aspartame, may be used. The proportion of natural carbohydrates in the composition is generally about 0.01 g to 0.04 g, and preferably about 0.02 g to 0.03 g per 100 g of the composition of the present invention.

The composition of the present invention may also contain various nutritional supplements, vitamins, electrolytes, flavoring agents, colorants, pectin and its salts, alginic acid and its salts, organic acids, protective colloidal agents, pH regulators, stabilizers, preservatives, glycerin, alcohol, and carbonation agents used in carbonated beverages.

Additionally, the composition of the present invention may contain fruit pulp for the production of natural fruit juices, fruit juice beverages, and vegetable drinks. These ingredients may be used independently or in combination. The proportion of these additives is not critically important, but typically, it is selected within the range of 0.01 to 0.1 parts by weight per 100 parts by weight of the composition of the present invention.

The present invention further relates to a feed additive for prevention or amelioration of tuberculosis including an extract of Philadelphus sp. plant as active ingredient.

The feed additive of the present invention corresponds to a supplementary feed according to the Feed Management Act. In the present invention, the term “feed” refers to any natural or artificial prescribed food, a meal, or components of a meal suitable for animals to consume, ingest, and digest. The type of feed is not particularly limited, and any feed commonly used in the relevant technical field may be used. Non-limiting examples of the feed include plant-based feed such as grains, roots and tubers, by-products of food processing, algae, fibrous materials, pharmaceutical by-products, oils, starches, oil cakes, or grain by-products; and animal-based feed such as proteins, minerals, oils, mineral substances, oils and fats, single-cell proteins, animal plankton, or food waste. These can be used either alone or in a mixture of two or more types thereof.

The present invention further relates to a veterinary composition for prevention or treatment of tuberculosis including an extract of Philadelphus sp. plant as active ingredient.

The veterinary composition of the present invention may further include appropriate excipients and diluents according to conventional methods. The excipients and diluents that may be included in the veterinary composition of the present invention include lactose, dextrose, sucrose, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methylhydroxybenzoate, propylhydroxybenzoate, talc, magnesium stearate, ethanol, stearyl alcohol, liquid paraffin, sorbitan monostearate, polysorbate 60, methylparaben, propylparaben, and mineral oil.

The veterinary composition according to the present invention may also further include fillers, anti-aggregation agents, lubricants, wetting agents, spices, emulsifiers, preservatives, and the like. The veterinary composition according to the present invention can be formulated using methods that are well-known in the art to provide rapid, sustained, or delayed release of the active ingredient after being administered to the animal. The formulation can take the form of powders, granules, tablets, capsules, suspensions, emulsions, solutions, syrups, aerosols, soft or hard gelatin capsules, suppositories, sterilized injectable solutions, sterilized topical formulations, and the like.

The effective amount of the veterinary composition according to the present invention can be appropriately determined based on the individual animal. It may be determined by factors such as the severity of the disease or condition, the animal's age, weight, health status, or sensitivity to the active ingredient based on sex, administration route, duration of administration, and other factors including compositions that are combined or used simultaneously with the composition of the present invention, as well as other elements well-known in the fields of physiology and veterinary science.

The present invention further relates to an oriental herbal medicine composition for anti-tuberculosis including an extract of Philadelphus sp. plant as active ingredient.

The oriental herbal composition of the present invention refers to one that is prepared according to prescriptions of traditional Korean medicine, but it is not limited thereto. An oriental herbal formulation including the oriental herbal composition according to one embodiment of the present invention may additionally be provided. The oriental herbal formulation only needs to be manufactured to include the oriental herbal composition, and the specific formulation is not limited. For example, the oriental herbal formulation may be in the form of a liquid, pill, tablet, granule, capsule, health food, or health beverage.

The present invention still further relates to a method for suppressing growth of Mycobacterium tuberculosis by administering an extract of Philadelphus sp. plant alone or in combination with an anti-tuberculosis agent to an animal other than human.

Hereinafter, the present invention will be described in greater detail with reference to the following examples. However, the examples are provided solely to illustrate more specifically the present invention, and it will be apparent to those having common knowledge in the pertinent art that the scope of the present invention is not limited thereby.

### Preparation Example 1. Preparation of Extract of Philadelphus sp. Plant

For 1 kg of dried Philadelphus tenuifolius leaves, 15 L of 70% (v/v) ethanol was added, followed by extraction at 85° C. for 3 hours and filtration. The resulting filtrate was then concentrated under reduced pressure and dried at 45° C. to give a Philadelphus tenuifolius leaf extract.

Meanwhile, leaves of Philadelphus schrenkii, Philadelphus pekinensis Rupr., Philadelphus seoulensis, and Philadelphus schrenckii var. jackii Koehne, all belonging to Philadelphus schrenkii sp., were extracted using ethanol in the same manner as described above for Philadelphus tenuifolius to give an extract of each plant.

### Example 1. Determination of Mycobacterium tuberculosis-Reducing Effect of Treatment with Extract of Philadelphus sp. Plant and Anti-Tuberculosis Agent in Macrophages Infected with Mycobacterium Tuberculosis

To measure the effect of the extract of Philadelphus sp. plant and an anti-tuberculosis agent on changes in the number of Mycobacterium tuberculosis within macrophages, bone marrow-derived macrophages (BMDMs) were infected with H37Rv, after which the extract of Philadelphus sp. plant and the anti-tuberculosis agent were respectively added, and the colony counts of the bacilli were measured.

Specifically, BMDMs isolated from the bone marrow of 6-week-old female C57BL/6 mice were counted, and then infected with pathogenic Mycobacterium tuberculosis H37Rv such that MOI=1. After 3 hours, the culture medium was replaced with a medium containing either the extract of Philadelphus sp. plant (200 μg/mL) or the anti-tuberculosis agent (100 ng/mL), followed by incubation for 48 hours at 37° C. under 5% CO₂. Subsequently, 500 μL of sterile triple-distilled water was added to each well to collect intracellular Mycobacterium tuberculosis, and serial dilutions were carried out tenfold up to 100-fold. To measure viable intracellular Mycobacterium tuberculosis, 10 μL aliquots were plated onto mycobacteria-selective medium (7H10 medium) containing ampicillin (25 ng/mL) and cultured for 2 weeks at 37° C. Thereafter, the number of intracellular Mycobacterium tuberculosis was determined by CFU (colony-forming unit) analysis.

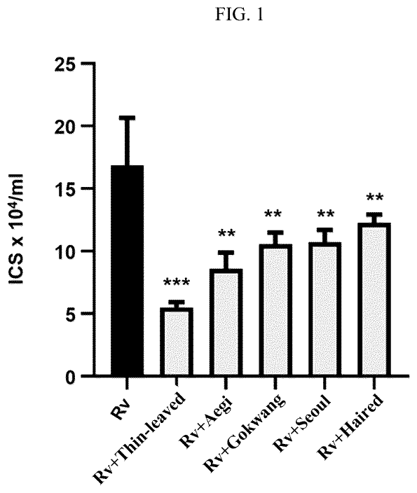

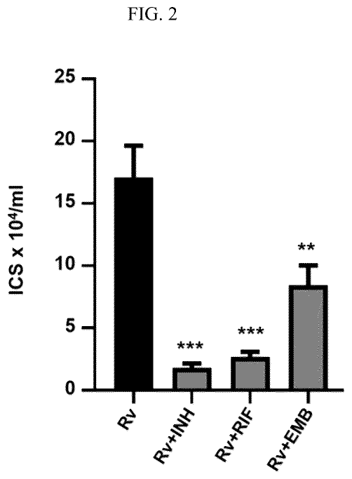

As a result, as illustrated in FIGS. 1 and 2, it was shown that both the group treated with the extract of Philadelphus sp. plant and the group treated with the anti-tuberculosis agent exhibited excellent inhibitory effects on the proliferation of Mycobacterium tuberculosis.

### Example 2. Determination of Mycobacterium tuberculosis-Reducing Effect of Treatment with Philadelphus tenuifolius Leaf Extract Alone or in Combination with an Anti-Tuberculosis Agent in Macrophages Infected with Mycobacterium Tuberculosis

Bone marrow-derived macrophages (BMDMs) isolated from the bone marrow of 6-week-old female C57BL/6 mice were counted, and then infected with pathogenic Mycobacterium tuberculosis H37Rv such that MOI=1. Three hours post-infection, the medium was replaced with a medium containing Philadelphus tenuifolius leaf extract at varying concentrations (50 μg/mL) and/or anti-tuberculosis agent (rifampicin (0.1 μg/mL), isoniazid (0.1 μg/mL), or ethambutol (4 μg/mL)). The cells were then incubated for 48 hours at 37° C. under 5% CO₂, after which the number of viable intracellular Mycobacterium tuberculosis was measured to determine the minimal inhibitory concentration (MIC). The MIC was defined as the lowest concentration that inhibited more than 99% of bacterial growth.

The fractional inhibitory concentration (FIC) value for quantifying the interaction between the Philadelphus tenuifolius leaf extract and anti-tuberculosis agent was calculated according to Equation 1 below, and the criteria for interpreting the resulting values are presented in Table 1.

$$FIC value ( \sum FICindex ) = FICB + FICA ( A = Philadelphus tenuifolius extract , B = Anti - tuberculosis agent ) \qquad [ Equation 1 ]$$

FICRMP=MICB combination/MICB alone
FICA=MICA combination/MICA alone

TABLE 1

| FIC value (x) | Effect |
|---|---|
| x ≤ 0.5 | Synergistic |
| 0.5 < x ≤ 0.75 | Partially synergistic |
| 0.75 < x ≤ 1.0 | Additive |
| 1.0 < x ≤ 4.0 | Non-interactive |
| x > 4.0 | Antagonistic |

As a result of quantifying the interaction between the Philadelphus tenuifolius leaf extract and anti-tuberculosis agent, it was found, as shown in Table 2 below, that when the Philadelphus tenuifolius leaf extract was used alone, the minimal inhibitory concentration (MIC) was 50 μg/mL. When rifampicin and isoniazid were each used alone, the MIC was 0.1 μg/mL, and when ethambutol was used alone, the MIC was 4 μg/mL. However, when they were used in combination, the MIC values were 6.25 μg/mL for the Philadelphus tenuifolius leaf extract, 0.03 μg/mL for rifampicin and isoniazid, and 0.3 μg/mL for ethambutol, thereby showing a synergistic effect. Accordingly, when the Philadelphus tenuifolius leaf extract is co-administered with an anti-tuberculosis agent, the synergistic effect between the two substances enhances the anti-tuberculosis activity of the Philadelphus tenuifolius leaf extract and increases the drug sensitivity of an anti-tuberculosis agent, specifically rifampicin, isoniazid, and ethambutol, against Mycobacterium tuberculosis.

TABLE 2
Result of examining Mycobacterium tuberculosis-reducing effect of treating
Rv-infected BMDM cells with mixture of anti-tuberculosis agent (rifampicin, isoniazid,
or ethambutol) and Philadelphus tenuifolius leaf extract

| | MIC alone | | | | | |
|---|---|---|---|---|---|---|
| | | Philadelphus | MIC combination | | | |
| | | tenuifolius | | Philadelphus | | |
| | | leaf | | tenuifolius | | Mycobacterium |
| | Antibiotics | extract | RIF | leaf extract | FIC | tuberculosis- |
| | (μg/ml) | (μg/ml) | (μg/ml) | (μg/ml) | value | reducing effect |
| Philadelphus | 0.1 | 50 | 0.03 | 6.25 | 0.43 | Synergistic |
| tenuifolius leaf | | | | | | |
| extract + rifampicin | | | | | | |
| (RIF) | | | | | | |
| Philadelphus | 0.1 | 50 | 0.03 | 6.25 | 0.43 | Synergistic |
| tenuifolius leaf | | | | | | |
| extract + isoniazid | | | | | | |
| (INH) | | | | | | |
| Philadelphus | 4 | 50 | 0.3 | 6.25 | 0.2 | Synergistic |
| tenuifolius leaf | | | | | | |
| extract + ethambutol | | | | | | |
| (EMB) | | | | | | |

### Example 3. Measurement of Mycobacterium tuberculosis Counts after Administration of Philadelphus tenuifolius Leaf Extract to H37Rv-Infected Mice

To examine the effect of the Philadelphus tenuifolius leaf extract on intracellular Mycobacterium tuberculosis in vivo, mice were infected with H37Rv and then administered with the Philadelphus tenuifolius leaf extract for 2 weeks, after which the colony counts of Mycobacterium tuberculosis in the lungs were determined.

More specifically, eight 7-week-old female C57BL/6 mice (19 g) were infected via the intratracheal route with pathogenic Mycobacterium tuberculosis (10⁶/50 μL). The infected mice were divided into two groups: a negative control group, which received 0.5% CMC (carboxymethylcellulose) as a vehicle, and the test group, which received the Philadelphus tenuifolius leaf extract (100 mg/kg). During the test, the Philadelphus tenuifolius leaf extract was used after dissolving it in 0.5% CMC. Starting from day 3 post-infection, the extract was orally administered five times at 2-day intervals. On day 14 post-infection, the mice were sacrificed, and the lungs were isolated and homogenized using a tissue homogenizer. The homogenates were serially diluted tenfold in 1 mL of sterile triple-distilled water, and 10 μL of each dilution was plated onto Mycobacterium tuberculosis-selective medium (7H10 medium) containing ampicillin (25 ng/mL). The plates were incubated at 37° C. for 2 weeks, and the number of bacteria in the tissue was determined by CFU (colony-forming unit) assay.

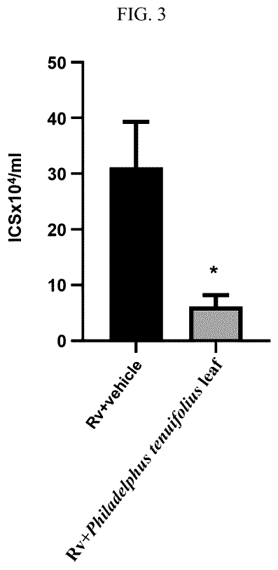

As a result, as illustrated in FIG. 3, the test group treated with the Philadelphus tenuifolius leaf extract exhibited a significant reduction in the number of viable Mycobacterium tuberculosis within lung cells compared to the negative control group.

### Statistical Analysis

All statistical analyses in the above Examples were performed using GraphPad Prism 8 (GraphPad, Inc., San Diego, CA), and significant differences between two groups were determined using the Mann-Whitney test (P values: ***p<0.001, **p<0.01, and *p<0.05).

1.-13. (canceled)

14. A method for suppressing growth of Mycobacterium tuberculosis, the method comprising:
administering a composition comprising an extract of Philadelphus sp. plant to an animal other than human.

15. The method of claim 14, wherein the composition further comprises an anti-tuberculosis agent.

16. The method of claim 15, wherein the anti-tuberculosis agent is one or more selected from rifampicin, isoniazid, and ethambutol.

17. The method of claim 14, wherein the extract of Philadelphus sp. plant is one or more selected from an extract of Philadelphus tenuifolius leaf, an extract of Philadelphus pekinensis Rupr. leaf, an extract of Philadelphus schrenkii leaf, an extract of Philadelphus seoulensis leaf, and an extract of Philadelphus schrenckii var. jackii Koehne leaf.

18. The method of claim 14, wherein the extract of Philadelphus sp. plant is extracted using water, C₁ to C₄ lower alcohol, or a mixture thereof as an extraction solvent.

19. The method of claim 14, wherein the composition is a pharmaceutical composition comprising (i) the an extract of Philadelphus sp. plant and (ii) a pharmaceutically acceptable carrier, an excipient, and/or a diluent.

20. The method of claim 14, wherein the composition is a functional health food composition formulated in any one form selected from the group consisting of powder, granule, pill, tablet, capsule, candy, syrup, and beverage.

21. A method for treating tuberculosis, the method comprising:
administering a composition comprising an extract of Philadelphus sp. plant to a subject in need thereof.

22. The method of claim 21, wherein the composition further comprises an anti-tuberculosis agent.

23. The method of claim 22, wherein the anti-tuberculosis agent is one or more selected from rifampicin, isoniazid, and ethambutol.

24. The method of claim 21, wherein the extract of Philadelphus sp. plant is one or more selected from an extract of Philadelphus tenuifolius leaf, an extract of Philadelphus pekinensis Rupr. leaf, an extract of Philadelphus schrenkii leaf, an extract of Philadelphus seoulensis leaf, and an extract of Philadelphus schrenckii var. jackii Koehne leaf.

25. The method of claim 21, wherein the extract of Philadelphus sp. plant is extracted using water, C₁ to C₄ lower alcohol, or a mixture thereof as an extraction solvent.

26. The method of claim 21, wherein the composition is a pharmaceutical composition comprising (i) the an extract of Philadelphus sp. plant and (ii) a pharmaceutically acceptable carrier, an excipient, and/or a diluent.

27. The method of claim 21, wherein the composition is a functional health food composition formulated in any one form selected from the group consisting of powder, granule, pill, tablet, capsule, candy, syrup, and beverage.

28. The method of claim 21, wherein the subject is an animal, and the composition is a veterinary composition comprising the extract of Philadelphus sp. plant and an anti-tuberculosis agent.
